(19) United States
(12) Patent Application Publication
(10) Pub. No.: US 2015/0284862 A1
(43) Pub. Date: Oct. 8, 2015

(54) Continuous Flow Process for Producing Storable Consumable Magnascent Iodine

(71) Applicant: John Staley Brookshire, Azle, TX (US)

(72) Inventor: John Staley Brookshire, Azle, TX (US)

(21) Appl. No.: 14/246,818

(22) Filed: Apr. 7, 2014

(51) Int. Cl. C25B 1/24; A61K 33/18

(52) U.S. Cl. CPC C25B 1/24; A61K 33/18

(57) ABSTRACT

Shown is a continuous method for the production of storable, consumable nascent iodine. A quantity of tincture of iodine solution is run in a continuous flow path through an electrolytic bath contained in the interior of a reactor vessel. Diatomically bonded iodine molecules in the tincture of iodine solution passing through the continuous flow path are subjected to an electromagnetic field created in the electrolytic bath by a pair of electrodes contained in the electrolytic bath in the reactor vessel. The tincture of iodine solution is subjected to a predetermined current density for a predetermined period of time, the current density being sufficient to decompose the diatomically bonded iodine molecules into elemental, pre-nascent iodine.

### BACKGROUND OF THE INVENTION

1. Field of the Invention

The present invention relates generally to a continuous process for producing storable, consumable, Magnascent iodine in a tincture which is used as an iodine supplement. The resulting product has sufficient iodine in an electromagnetically excited state to be active in the body for 2 to 3 hours as nascent iodine.

2. Description of the Prior Art

Nascent iodine is a well-known anti-infective agent as well as a vital component of T3 and T4 hormone production within the body. Attempts to find an effective method for ingesting nascent iodine began with the use of Brown Iodide of Lime, as a loose combination of “iodine” and calcium oxide, which sets nascent iodine free when taken into the stomach, originated by Nichols (A Practitioner's Handbook Of Materia Medica And Therapeutics Based Upon Established Physiological Actions by Thomas S. Blair, M.D., 1907.) This was not a very effective method even at that time.

Today there are several patents utilizing nascent iodine as the primary factor. One set of patents (U.S. Pat. No. 6,838,050 and U.S. Pat. No. 6,482,309) refers to the electrolytic generation of nascent iodine as a method of treatment for the prevention of infections associated with medical implant devices. Another patent (U.S. Pat. No. 5,232,914) relates to the use of nascent iodine in a solid germicidal, pre-iodine composition. Still another (U.S. Pat. No. 6,592,890) refers to use of nascent iodine in a wound dressing. And the last relevant patent known to Applicant (U.S. Pat. No. 5,538,740) refers to the therapeutic and cosmetic compositions involving nascent iodine for the treatment of skin. None of the above patents involve a consumable form of nascent iodine.

In 1912 Sunkar A. Bisey developed a Westernized form of nascent iodine. This formula was in a saline solution, based upon traditional medicines from India. This traditional medicine was utilized to treat fevers, malaria, and other illnesses, and was produced primarily from seaweed. No patent for Bisey's product has been located, (although Bisey had several patents). However, information obtained from Schieffelin and Company indicates that this formula was highly utilized by physicians and hospitals from 1926 until Bisey's death in 1935. Promotional claims by Schieffelin stated that this saline/iodine solution was active in the nascent state when dissolved in water and consumed (Druggist's circular titled “Atomidine in Medicine”, distributed by Schieffelin & Co. Mount Vernon, N.Y.). This circular refers to the 1930's product only. Although no record was found of the exact composition of the saline solution (other than the iodine portion), information was obtained about a process which was utilized to produce this form of nascent iodine.

In October 1931, Dr. Sunkar A. Bisey met with Edgar Cayce of Virginia Beach, Va., and an investor interested in Bisey's nascent iodine product. Edgar Cayce suggested a change in the method of production, and this new method of production formed the basis for a saline iodine solution which Bisey used for the last four years of his life. A tincture of iodine solution has obvious differences with a saline solution, but since both allegedly produced nascent iodine indicate there should be similar characteristics. After Bisey's death in 1935, the use of his product seems to have declined and then disappeared.

There is one company (previously two) that claims to produce the Bisey product today, but it is based on iodine trichloride and has not until recently claimed to present nascent iodine to the body system; in fact, it evenwarns against internal use. This company holds its production methods in secret; all that is known is that the product is 1% iodine trichloride, which is considered toxic in quantities larger than a few drops.

In the late 1990's Phillip Thomas of Altamonte Springs, FL, experimented with a 1% tincture of iodine and achieved a product he called Rejuvidine™. It was produced by a method similar to Sunkar A. Bisey's product. It provided a significant step forward as a health supplement, but due to several factors, the product did not achieve a sufficient energy level to attain the nascent state of iodine in useful quantity.

The need for a safe iodine supplement. Due to the nature of iodine and the fear of iodine toxicity, it is essential that as much of the iodine be in a nascent state as possible because the human body should not just consume more and more iodine at a time to gain the nascent iodine benefit. Hence, a method to achieve a high quantity of nascent state iodine is desired. This need has never been greater according to those researching iodine deficiencies. Dr. David Brownstein, author of Iodine Why You Need It, Why You Can't Live Without It, reports that 95% of his patients tested have demonstrated low iodine levels.

Applicant's issued U.S. Pat. No. 8,323,703, issued Dec. 4, 2012, describes a batch process for the production of storable, pre-nascent iodine that activates and produces nascent iodine over a two to three hour period within the body when diluted in water and consumed. While the teaching of the '703 patent represents an advance in the art of producing consumable nascent iodine, there was a need for an improved method, particularly a production method, which would involve a continuous flow, as opposed to the batch method described in the '703 patent.

A need continued to exist, therefore, for an improved production method for producing nascent iodine, which method more effectively produces nascent iodine through a continuous flow process with resulting improved consistency of product, with an improved environmental effect, and with fewer man-hours required in the method.

### SUMMARY OF THE INVENTION

In the method of the present invention, a tincture of iodine solution is subjected to an electromagnetic current as it flows through a non-permeable tube in an electrolytic bath, i.e. maker solution, to produce nascent iodine in a form which is useful to the human body, now referred to as Magnascent® iodine according to prior patent U. S. Pat. No. 8,323,703 but is now produced using a continuous flow process instead of a batch process

The nascent iodine level may become activated when diluted in water and consumed, and the nascent iodine may form within the body as the iodine gradually loses energy (relaxes) when diluted.

The nascent iodine may perform anti-infective activity, and the nascent iodine may act as a catalyst to produce T3 and T4 hormones within the thyroid.

The nascent iodine may be applicable to fight parasites including malaria, dengue, and black-water fever within the body, and the nascent iodine may be adaptable to fight gastro-intestinal disorders.

The nascent iodine may be applicable to reduce blood pressure, and the nascent iodine may be adaptable to treat iodine deficiency diseases.

The nascent iodine may be applicable to treat sinusitis, bronchitis, and asthma, and step of raising the energy level may include breaking the diatomic bond of the iodine.

The general method of this invention is a continuous flow method for the production of storable, consumable nascent iodine. The method includes the steps of:

flowing through a tube tincture of iodine solution through an electrolytic bath where the iodine in the tincture of iodine solution is initially in the form of diatomic iodine molecules;

providing a reactor vessel containing an electrolytic bath, having an interior resistant to acidic electrolytic solution, a fluid inlet to a long tube impermeable and resistant to degradation by electrolytic solution and by internal iodine tincture, and a fluid outlet;

passing the tincture of iodine solution through the tube in the interior of the reactor vessel in a continuous flow path between the fluid inlet and the fluid outlet to a collection tank for the resultant Magnascent® iodine tincture after the diatomiciodine molecules have been subjected to the magnetic field.

The electromagnetic field created in the electrolytic bath is created by at least a pair of electrodes using a predetermined current density for a predetermined period of time, the current density being sufficient to decompose and excite the diatomic iodine molecules into both diatomic yet energetically excited pre-nascent iodine and monoatomic nascent iodine, i.e. Magnascent® iodine. Pre-nascent iodine in Magnascent® iodine can be activiated by excess solvent and the body to form additional beneficial nascent iodine, and prenascent iodine is storable whereas nascent iodine degrades rapidly though exists in a small amount in equilibrium in the Magnascent® iodine tincture.

The preferred method of this invention is a continuous flow method for the production of storable, consumable nascent iodine with the following specific steps and parameters:

Iodine flows continuously in a serpentine path through a tube. There is at least one gallon of electrolytic bath solution for each ounce of tincture of iodine solution passing through the continuous flow path in the reactor vessel interior during the production process. The tube is of sufficient size to contain at least three gallons of tincture of iodine i.e. 384+ gallons of electrolytic solution as this is a high-throughput invention. The tube can conveniently be placed on racks located within the reactor vessel and runs for a total length between about 4 and 5 feet. Copper (Cu) and Nickel (Ni) rods of ½ inch diameter, approximately 26 inches long are supported on either of two opposite sides of the serpentine tubing and are used to generate the electromagnetic field. The rods are connected to an alternating current source and are inserted within the electrolytic bath. One or more set of rods are mounted vertically within the interior of the reactor vessel within the electrolytic bath and are movable up and down within the electrolytic bath until a current of approximately 30 amps is achieved within the electrolytic bath.

The preferred electrolytic bath is a copper sulfate/sulfuric acid/zinc/distilled water solution. A porous container, such as a basket, is supported within the interior of the reactor vessel and contains a quantity of zinc nodules. Additional zinc nodules may be added to adjust the concentration of zinc in the electrolytic bath.

A valve is preferably provided in a sidewall of the reactor vessel to allow distilled water to be added during the operation to make up for any evaporative loss during the operation of the reactor vessel and due to electrolysis of water from the electromagnetic field. To further control the temperature of the reactor vessel, in some circumstances, a set of cooling tubes flowing cool water are run parallel to the serpentine tube on either of two opposite sides thereof to provide heat reduction in the electrolytic bath. An exit port is preferably provided in an upper third portion of the reactor vessel to allow vapors from a gaseous tank area to be withdrawn and processed before being released back to the surrounding atmosphere.

The pre-nascent iodine in the Magnascent® iodine being produced from the tincture of iodine solution is in a concentration sufficient to make it useful to a human body and becomes activated in the form of nascent iodine when diluted in water and consumed.

Additional objects, features and advantages will be apparent in the written description which follows.

### BRIEF DESCRIPTION OF THE DRAWINGS

FIG. 1 is a simplified perspective view of a reactor vessel which is used in the continuous process for producing nascent iodine of the invention.

FIG. 2 is a top view of the vessel of FIG. 1.

FIG. 3 is a schematic view of the operation of the vessel of FIG. 1 in the continuous manufacturing process of the invention.

### DETAILED DESCRIPTION OF THE INVENTION

The preferred version of the invention presented in the following written description and the various features and advantageous details thereof are explained more fully with reference to the non-limiting examples and as detailed in the description which follows. Descriptions of well-known components and processes and manufacturing techniques are omitted so as to not unnecessarily obscure the principle features of the invention as described herein. The examples used in the description which follows are intended merely to facilitate an understanding of ways in which the invention may be practiced and to further enable those skilled in the art to practice the invention. Accordingly, the examples should not be construed as limiting the scope of the claimed invention.

In the method of the invention, a storable, consumable form of nascent iodine is produced that is effective in a safe quantity (a few drops) and yet has a strong nascent iodine component, promoting increased energy and improved health as a general health supplement. An electromagnetic field is used to produce the breaking of the diatomic bond of the iodine molecule through magnetic excitation. Tincture of iodine is an excellent medium for containing this state of pre-nascent iodine until activated in water and consumed. Other forms of production with continuous flowing tincture solution and using direct magnetic fields can be devised for industrial production. The body can utilize iodine in the nascent state in a natural way, since the nascent state of iodine is generated within the thyroid in order to produce T3 and T4 hormones. Supplementing the diet with iodide or iodine is known to boost the immune system. The addition of iodine in the nascent state with its well-documented anti-infective effect will complement this health benefit in the body. It is submitted at this time only as a health supplement.

As briefly discussed in the Background portion of the present Specification, Applicant's previously issued U.S. Pat. No. 8,323,703 (“the '703 patent”) describes a batch process for producing the nascent iodine, now referred to as Magnascent® iodine, which is the object of the presently to be described continuous production process. The batch process will be briefly described since it forms the basis for the improved continuous process which is the subject of the present invention.

Description of the Batch Process:

As has been described, in order to be of full benefit, the energy level of the raw diatomic iodine has to be increased in the tincture of iodine creating pre-nascent iodine before release of nascent iodine upon activation. The Rejuvidine™ (made by Phillip Thomas and described in the Background Section) is produced (16 ounces of tincture of iodine) by submerging it in an electrolytic bath of 3 gallons of maker solution for five minutes at a resistance of 10 amp of AC current. Applicant's previously described batch process i.e. Magnascent® iodine production process differed from the Rejuvidine™ process in the following ways: the amperage was increased from 10 amps to 30 amps; the quantity of tincture being energized was decreased from 16 ounces to eight ounces, while the maker solution (electrolytic solution) was increased from 3 gallons to 8 gallons, and the time was increased from 5 minutes to 20 minutes. This increased level of activity is sufficient to break the diatomic bond of the iodine and elevate the iodine atom into the nascent monoatomic state.

The resulting Magnascent® iodine produces sufficient nascent iodine upon activation to give the desired effect for 2-3 hours in the body using only a few drops dissolved in water and consumed. This quantity of nascent iodine required to have beneficial effects is similar or less than the quantity of iodine required to purify water for drinking in many areas of the world.

The simplest way to produce the nascent-reacting iodine in a batch process is to prepare a tincture of iodine, for example 1% iodine by weight in grain alcohol. Prepare an electrolytic bath of copper sulfate, sulfuric acid and zinc chips. (An effective electrolytic solution is ⅔ lb of copper sulfate per gal., ½ ounce of sulfuric acid per gal. and ⅓ ounce of zinc chips per gal.) Place anodes of copper and of nickel alloy (½ inch diameter and 14 inches long) attached to an AC current on either side of a glass container, with the tincture of iodine, submerged in the electrolytic bath. The AC current is run at 30 amps of resistance for 20 minutes. The volume of tincture of iodine in ounces should not exceed the gallons of the electrolytic solution. The container for the electrolytic solution should be glass, ceramic, plastic, or other material resistant to the acidic maker solution.

The nascent-reacting iodine so produced should be stored in a dark glass container at a cool temperature and out of direct light. It is most effective when consumed directly after mixing a few drops with at least 2 to 3 ounces of water and taken on an empty stomach. Best Mode For Making 8 Ounces of Nascent Iodine By the Batch Method:

1. A plastic container of sufficient size is filled with 8 gallons of distilled water, allowing for a few inches of clearance between the top of the water and top of the container.

2. An electrolytic solution is made using 5⅓ lb. of copper sulfate, 4 ounce of sulfuric acid and 2⅔ ounce of zinc chips. The chemicals are added in the previously described order and are mixed for approximately 20 minutes.

3. A ½ inch diameter copper rod of 14 inch length functions as an anode and is attached to the one side of the container. A similar size nickel rod is attached to the opposite side of the container as the other electrode. The amperage can be adjusted to maintain the necessary 30 amps by adjusting the depth that these rods extend into the electrolytic solution. While 30 amps is a particularly preferred current, in some situations the current may be generally in the range from about 20-40 amps.

4. A long necked glass flask with a glass stopper containing No more than 8 ounces of 1% tincture of iodine (up to 2.2% allowed by DEA) made from ethanol (grain alcohol, organic alcohol or other) is introduced into a long necked glass flask with a glass stopper. The glass flask is lowered into the center of the container so that the electromagnetic field surrounds the flask.

5. Using safe electrical procedures, a 110 volt AC current is connected to flow through the anodes across the electrolytic solution for 20 minutes, at 30 amps.

6. The tincture of iodine is now in the pre-nascent state and should be stored in a dark glass bottle to avoid photo degradation until ready for use.

7. A typical use as a health supplement for an iodine deficient person might be 5 drops in a half glass (4 ounces) of water. It is most effective taken on an empty stomach. It will be effective in the body in the nascent state for up to two or three hours as the excitation energy from the electromagnetic field relaxes. Consumable nascent iodine has provided the body with only 5 drops of 1% tincture of iodine, less iodine than would be used to purify a glass of water on a camping trip.

Description of the Improved Continuous Production Process:

As has been mentioned, while the teaching of the '703 patent represents an advance in the art of producing consumable nascent iodine, Magnascent® iodine, there continued to be a need for an improved production method which would operate as a continuous flow method, as opposed to the batch method described above. The continuous process, to be described in detail below, constitutes an improved production method for producing Magnascent® iodine, which method more effectively produces nascent iodine through a continuous flow process with resulting improved consistency of manufacturing and product, with an improved environmental effect, and with fewer man-hours required in the method.

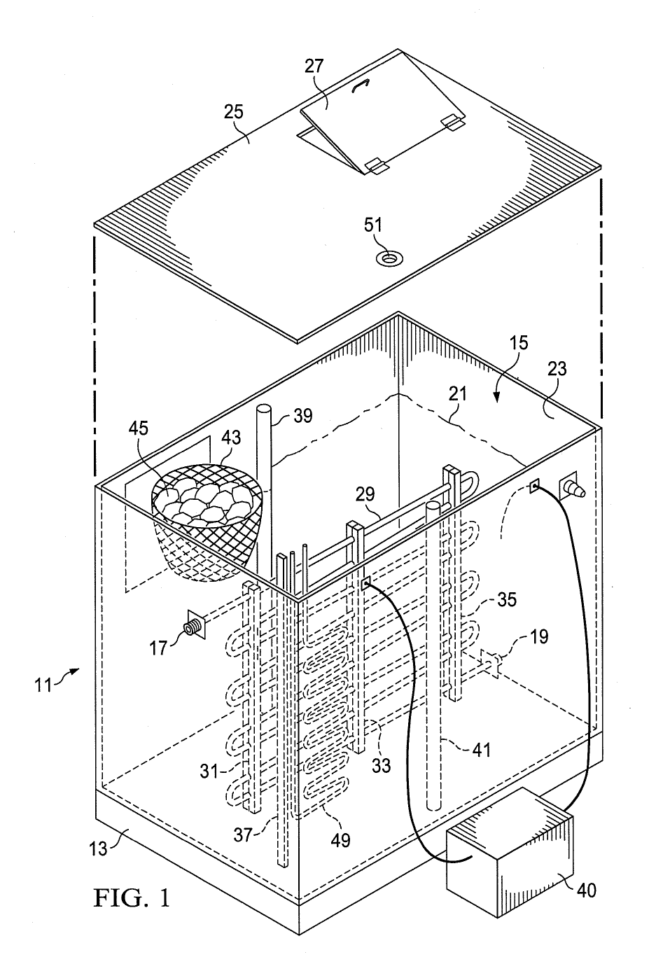

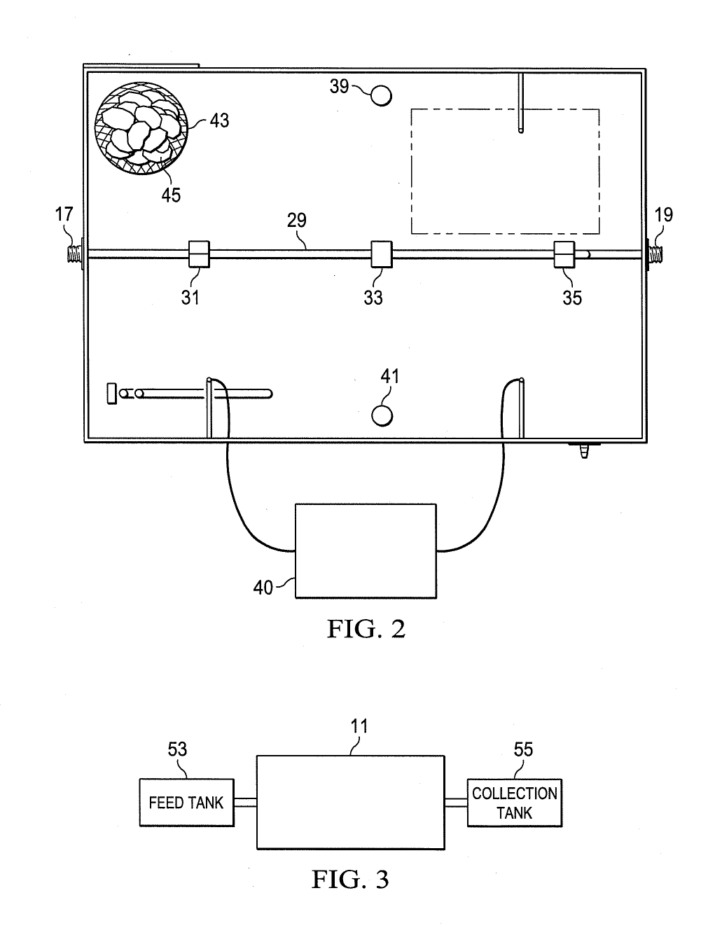

With reference now to FIG. 1 of the Drawings, there is shown an apparatus for use in the continuous flow process of the invention. A reactor vessel, in this case a large tank 11 of sufficient size, sits on a base 13 which is approximately 6 inches above the floor and which has a method to replace the electrolytic solution once time depleted. The tank is constructed of material as deemed suitable with appropriate drain plugs and attachments as required to fulfill the process. The vessel has an interior 15, a fluid inlet 17 and a fluid outlet 19. As will be described in detail, the vessel contains an electrolytic bath 21 which preferably comprises a copper sulfate, zinc, and dilute sulfuric acid solution. The interior sidewalls 23 of the vessel are highly resistant to the electrolytic bath filling approximately ⅔ of the tank.

A cover 25 of similar construction to the tank body is placed on top of the tank to ensure containment of vapors generated from the electromagnetic solution which are subsequently created. A small observation and entry door 27 may be located at one end of the cover.

A flexible tube 29 passes in a serpentine path between the fluid inlet 17 and the fluid outlet 19 within the interior of the reactor vessel 11. The tube is made of a resistant material so as not to be affected by the electrolytic bath solution in the tank, or the tincture of iodine solution which will subsequently pass through the interior of the tube. There are several important aspects to the tube, the first being that the diameter is of sufficient size to contain the desired gallons of tincture of iodine within the tube itself. This diameter may be, for example, 1 inch which would require the tube to be placed on racks running back and forth inside the tank for a distance of between 4 and 5 feet, in this example. A frame including components 31, 33, 35, is placed near the beginning, middle and end of the tubing to allow a turnaround of the tube and support of the tube. A non-reactive stand 37 will be built to support the frame components and the tubing. The frame components which would be located at approximately 6 inches from the ends of the tubing run, and in the middle of the tubing in this example.

Copper rods 39, 41 , of necessary size (approximately ½ inch in diameter, and approximately 26 inches long in this example) are placed on either side of the tubing and are connected to an AC power source (40 in FIG. 1) to generate the electromagnetic field. The rods 39, 41 can be located on either side or either end of the tank interior. They are supported by attachments (not shown) projecting from the sides of the tank. Electrical wire will be attached to these rods to power the electromagnetic field. A set of wires (not shown) will also be used to raise and lower the rods as required to achieve a 10-35 amp current (30 amp preferred).

A basket 43 holding zinc nodules, as described in the '703 patent, is located within the vessel interior. Additional zinc nodules, copper sulfate, sulfuric acid, and distilled water may be added to the basket or solution as required to adjust the copper sulfate/sulfuric acid/zinc/distilled water solution (Solution No. 1 as described in the '703 patent). The basket has the capability of being raised and lowered during operation. Of critical importance is that fact that there must be at least 1 gallon of Solution No. 1 for every ounce of iodine tincture in the tubing. There is no problem in exceeding the amount of Solution No. 1 per ounce of tincture.

An exit port 51 in the upper third of the tank wall will be used to withdraw and clean air from the gaseous tank area. It may be necessary to process this through water or use other conventional treatment techniques to remove any vapors prior to discharge.

The electromagnetic solution will be of the same ratio and be made from the same constituents as found in the '703 patent but volume of the tank containing this liquid will be much larger (hundreds of gallons) for example approximately 64 cubic feet or about 480 gallons in an exemplary set-up. In keeping with the ratio established in the '703 patent, of 1 gallon of electrolytic solution to 1 ounce tincture of iodine being put into the nascent state, this allows for as many ounces of tincture to the converted as contained in the tubing (3 gallons for example). This tincture is found in the tubing 29 that flows back and forth within the electromagnetic field for the time required to achieve the nascent state. Using a 1 inch diameter tube as an example, the requirement is approximately 300 inches length for each “run” of the tube, we would need five “runs” per gallon. This diameter may be modified to a smaller diameter with an increased in the number of runs required. The whole set-up is designed so that there is a continuous flow in the system and supports along the way insure the tubing stays in place, and that the time in the electromagnetic field is sufficient to convert the tincture of iodine to the nascent state.

As shown in FIG. 3, the overall system will have a feed tank 53 at the beginning of the process and a collection tank 55 after the processing with a time control to insure that the tincture stays within the system long enough within the electromagnetic field to put iodine into the nascent state.

Temperature measurements can be performed within the tank (in the electrolytic bath) to ensure that excessive heat is not building up. A cooling system may be required to ensure that the temperature stays within predetermined limits.

An invention has been provided with several advantages. The process of the invention produces a storable, consumable form of nascent iodine that can provide increased energy and improved health as a general health supplement. The continuous flow process of the invention provides improved production rates over the batch process described in Applicant's earlier patent. The improved method of the invention provides for the more effective production of nascent iodine, with more consistent quality, with an improved environmental effect, and also with fewer man-hours required for running the operation.

While the invention has been shown in only one of its forms, it is not thus limited but is susceptible to various changes and modifications without departing from the spirit thereof.

What is claimed is:

1. A continuous method for the production of storable, consumable nascent iodine, the method comprising the steps of:
supplying a given quantity of tincture of iodine solution in a feed tank, the iodine in the tincture of iodine solution being in the form of diatomically bonded iodine molecules;
providing a reactor vessel having an interior, a fluid inlet and a fluid outlet, the reactor vessel containing an electrolytic bath;
passing the tincture of iodine solution through the interior of the reactor vessel in a continuous flow path between the fluid inlet and the fluid outlet to a collection tank, the diatomically bonded iodine molecules in the tincture of iodine solution passing through the continuous flow path being subjected to an electromagnetic field created in the electrolytic bath by at least a pair of electrodes contained in the electrolytic bath in the reactor vessel, the tincture of iodine solution being subjected to a predetermined current density for a predetermined period of time, the current density being sufficient to decompose at least a portion of the diatomically bonded iodine molecules into elemental, pre-nascent iodine.

2. The method of claim 1, wherein the tincture of iodine solution passes in a serpentine path through a tube of a predetermined diameter and a predetermined length within the interior of the reactor vessel between the fluid inlet and the fluid outlet.

3. The method of claim 2, wherein the tube is of sufficient size to contain at least about three gallons of tincture of iodine within the tube itself.

4. The method of claim 3, wherein the tube is placed on racks located within the reactor vessel and runs for an extended length.

5. The method of claim 1, wherein copper rods of a certain size (for example ½ inch diameter, approximately 26 inches long) are supported on either of two opposite sides of the serpentine tubing and are used to generate the electromagnetic field.

6. The method of claim 5, wherein the copper rods are mounted within the interior of the reactor vessel within the electrolytic bath and are movable up and down within the electrolytic bath until a current of approximately 30 amps or similar amperage (10-35 amps) is achieved within the electrolytic bath.

7. The method of claim 1, wherein a porous container is supported within the interior of the reactor vessel and contains a quantity of zinc nodules, and wherein additional zinc nodules are added to adjust the concentration of zinc in the electrolytic bath.

8. The method of claim 7, wherein the electrolytic bath is a copper sulfate/sulfuric acid/zinc/distilled water solution, wherein copper sulfate, sulfuric acid, and distilled water can also be adjusted as required.

9. The method of claim 8, wherein there is approximately one gallon or more of electrolytic bath solution for each ounce of tincture of iodine solution passing through the continuous flow path in the reactor vessel interior.

10. The method of claim 1, wherein an exit port is provided in a portion of the reactor vessel to allow vapors from a gaseous tank area to be withdrawn and processed before being released back to the surrounding atmosphere.

11. The method of claim 1, wherein the pre-nascent iodine being produced from the tincture of iodine solution is in a concentration sufficient to make it useful to a human body.

12. The method of claim 11, wherein the pre-nascent iodine in solution which is produced becomes activated in the form of nascent iodine when diluted in water and consumed.

13. The method of claim 8, wherein the electrolytic bath is formulated by mixing copper sulfate, sulfuric acid and zinc chips in distilled water according to the following or a similar proportion to the following or using similar chemistry thereof: ⅔ pounds of copper sulfate per gallon of bath, ½ ounces of sulfuric acid per gallon of bath, and ⅓ ounces of zinc chips per gallon of bath.

14. The method of claim 5, wherein the electrodes of copper are connected to an alternating current source and are inserted within the electrolytic bath.
